(19) United States
(12) Patent Application Publication
(10) Pub. No.: US 2025/0382549 A1
(43) Pub. Date: Dec. 18, 2025

(54) MULTIFUNCTIONAL N-OXIDE HYDROTROPES, CLEANING FORMULATIONS CONTAINING THEM AND USE THEREOF

(71) Applicant: NOURYON CHEMICALS INTERNATIONAL B.V., Amsterdam (NL)

(72) Inventors: Hanamanthsa Shankarsa BEVINAKATTI, Somerset, NJ (US); Karen Lee WHITE, Bridgewater, NJ (US); Sorel MURESAN, Stenungsund (SE)

(73) Assignee: NOURYON CHEMICALS INTERNATIONAL B.V., Amsterdam (NL)

(21) Appl. No.: 18/878,368

(22) PCT Filed: Jun. 23, 2023
(86) PCT No.: PCT/EP2023/067090

§ 371 Date: Dec. 23, 2024

Related U.S. Application Data
(60) Provisional application No. 63/355,182, filed on Jun. 24, 2022.

(51) Int. Cl. C11D 1/825; C08G 65/324; C08G 65/333; C11D 1/722; C11D 1/75

(52) U.S. Cl. CPC C11D 1/8255; C08G 65/324; C08G 65/33306; C11D 1/722; C11D 1/75

(57) ABSTRACT

An N-oxide compound has the formula (I):

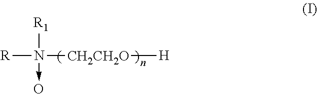

wherein
R represents a fatty linear or branched, saturated or unsaturated alkyl group having 8-30 carbon atoms;
R¹ represents a linear or branched, saturated or unsaturated lower alkyl group having 1-8 carbon atoms; and
n represents at least 8 and at most 25.
The compound of formula (I) acts as a hydrotrope and is useful for cleaning applications. Processes of preparing the compound, cleaning compositions containing the compound, and methods of using the compound to clean an object are also disclosed.

### CROSS-REFERENCE TO RELATED APPLICATIONS

This application is a U.S. National-Stage entry under 35 U.S.C. § 371 based on International Application No. PCT/EP2023/067090, filed Jun. 23, 2023, which was published under PCT Article 21(2) and which claims priority to U.S. Provisional Application No. 63/355,182, filed Jun. 24, 2022, which are all hereby incorporated in their entirety by reference.

### FIELD OF THE DISCLOSURE

The present disclosure relates to multifunctional hydrotropes and the use thereof in cleaning applications.

### BACKGROUND

As is well-known in the art, many surfactants are too hydrophobic to be soluble in water. Attempts to introduce such surfactants to water can result in cloudy or hazy solutions. In order to solubilize such surfactants, typically a hydrotrope must be added.

Hydrotropes in use include amphoteric surfactants and quaternary ammonium compounds as well as nonionic surfactants such as highly ethoxylated fatty acids and alkyl glucosides.

Quaternary ammonium compounds in use as hydrotropes include those, for example, described in U.S. Pat. No. 8,709,169 and European Patent No. 1 838 826. Compounds of this type are available from Nouryon (under the tradename BEROL®). However, quaternary ammonium salts are coming under increasing environmental pressure due to their safety and toxicity concerns and products of this class require labelling.

It is an object of the present disclosure to develop a bio-based multifunctional hydrotrope with low or no environmental persistence and ecotoxicity for cleaning formulations. In addition, other objects, desirable features and characteristics will become apparent from the subsequent summary and detailed description, and the appended claims, taken in conjunction with the accompanying drawings and this background.

### BRIEF SUMMARY

The present disclosure relates in one embodiment to a compound of the formula (I):

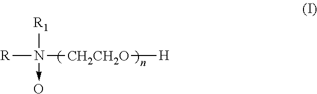

wherein
R represents a fatty linear or branched, saturated or unsaturated alkyl group having 8-30 carbon atoms;
R¹ represents a linear or branched, saturated or unsaturated lower alkyl group having 1-8 carbon atoms;
n represents at least 8 and at most 25.

Surprisingly, it has been found that compounds of the formula (I) are very efficient hydrotropes for nonionic surfactants, and also aid in the cleaning performance of compositions where they are present in combination with nonionic surfactants. In addition, compounds of the formula (I) are readily biodegradable and are expected to exhibit low/no toxicity.

Accordingly, the present disclosure relates in a second embodiment to an aqueous cleaning composition comprising:
(a) at least one compound of the formula (I):

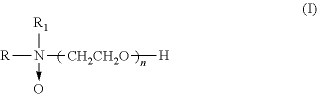

wherein
R represents a fatty linear or branched, saturated or unsaturated alkyl group having 8-30 carbon atoms;
R¹ represents a linear or branched, saturated or unsaturated lower alkyl group having 1-8 carbon atoms; and
n represents at least 8 and at most 25; and
(b) at least one nonionic surfactant.

The present disclosure relates in another embodiment to a process for preparing a compound of the formula (I), said process comprising:
(a) ethoxylating an amine of the formula (II):

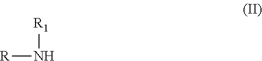

wherein
R represents a fatty linear or branched, saturated or unsaturated alkyl group having 8-30 carbon atoms; and
R¹ represents a linear or branched, saturated or unsaturated lower alkyl group having 1-8 carbon atoms;

to form an ethoxylated amine of the formula (III):

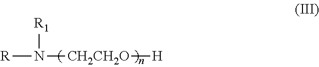

wherein R and R¹ have the meanings given above; and n represents at least 8 and at most 25; and
(b) oxidizing the ethoxylated amine of the formula (III) to give the N-oxide of formula (I).

The present disclosure relates in another embodiment to an aqueous cleaning composition comprising:
(a) at least one compound of formula (I) as described herein; and
(b) water.

The present disclosure relates in yet another embodiment to a method of cleaning an object to be cleaned comprising contacting the object with an aqueous cleaning composition as described herein.

### BRIEF DESCRIPTION OF THE DRAWINGS

The disclosure will now be described in greater detail with reference to the drawings, wherein:

FIG. 1A is an illustration depicting the cleaning power of Formulations A and C according to the present disclosure without NaOH on a painted steel panel soiled with engine grease; and

FIG. 1B is an illustration depicting the cleaning power of Formulations D and F according to the present disclosure in the presence of 2% NaOH on a painted steel panel soiled with engine grease.

### DETAILED DESCRIPTION

The following detailed description is merely exemplary in nature and is not intended to limit any composition. Furthermore, there is no intention to be bound by any theory presented in the preceding background or the following detailed description. It is to be appreciated that all numerical values as provided herein, save for the actual examples, are approximate values with endpoints or particular values intended to be read as “about” or “approximately” the value as recited.

Embodiments of the present disclosure are generally directed to compounds described above, compositions including the same, and methods for forming the same. For the sake of brevity, conventional techniques related to making such compounds and such compositions may not be described in detail herein. Moreover, the various tasks and process steps described herein may be incorporated into a more comprehensive procedure or process having additional steps or functionality not described in detail herein. In particular, various steps in the manufacture of such compounds and associated compositions are well-known and so, in the interest of brevity, many conventional steps will only be described briefly herein or will be omitted entirely without providing the well-known process details.

In this disclosure, the terminology “about” can describe values ±0.1, 0.5, 1, 2, 3, 4, 5, 6, 7, 8, 9, or 10%, in various embodiments. Moreover, it is contemplated that, in various non-limiting embodiments, it is to be appreciated that all numerical values as provided herein, save for the actual examples, are approximate values with endpoints or particular values intended to be read as “about” or “approximately” the value as recited.

The compounds and compositions disclosed herein may suitably comprise, consist of, or consist essentially of the components, elements, and process delineations described herein. The embodiments illustratively disclosed herein suitably may be practiced in the absence of any element which is not specifically disclosed herein.

In the compound of formula (I):
R represents typically C₈-C₂₂ alkyl or alkenyl, more typically C₈-C₂₀ alkyl or alkenyl, and most typically C₁₀-C₁₈ alkyl or alkenyl;
R¹ represents C₁-C₄ alkyl, more typically methyl or ethyl, and most typically methyl; and
n represents typically at least 9, and most typically at least 10, and typically at most 20, and most typically at most 17.

In various non-limiting embodiments, all values and ranges thereof, both whole and fractional, including and between those set forth above, are hereby expressly contemplated for use herein.

In a typical embodiment, the compound of formula (I) derives from a secondary amine of the formula (II):

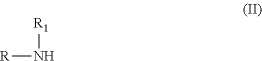

wherein
R represents a fatty linear or branched, C₈-C₂₀ alkyl or alkenyl, and most typically C₁₀-C₁₈ alkyl or alkenyl; and
R¹ represents a linear or branched, C₁-C₄ alkyl, more typically methyl or ethyl, and most typically methyl.

In various non-limiting embodiments, all values and ranges thereof, both whole and fractional, including and between those set forth above, are hereby expressly contemplated for use herein.

In an especially typical embodiment, the secondary amine is selected from the group consisting of octylmethylamine, cocoalkylmethylamine, laurylmethylamine, n-decylmethylamine, tallowalkylmethylamine, soyaalkylmethylamine, oleylalkylamine and C12/14alkylmethylamine.

In a more typical embodiment, the compound of formula (I) has the formula:

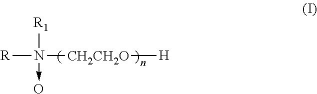

wherein
R represents a fatty linear or branched, C₁₀-C₁₈ alkyl or alkenyl;
R represents methyl or ethyl; and
n represents 8-17.

In various non-limiting embodiments, all values and ranges thereof, both whole and fractional, including and between those set forth above, are hereby expressly contemplated for use herein.

In a most typical embodiment, the compound of the formula (I) has the formula (Ia):

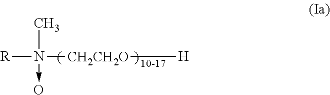

wherein
R represents a fatty linear or branched, saturated or unsaturated alkyl group having 8-14 carbon atoms.

In various non-limiting embodiments, all values and ranges thereof, both whole and fractional, including and between those set forth above, are hereby expressly contemplated for use herein.

As noted above, the compound of the formula (I) is prepared by a process comprising:
(a) ethoxylating an amine of the formula (II):

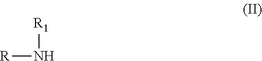

wherein
R represents C₈-C₂₂ hydrocarbyl, typically C₈-C₂₂ alkyl or alkenyl, more typically C₈-C₂₀ alkyl or alkenyl, and most typically C₁₀-C₁₈ alkyl or alkenyl; and
R¹ represents C₁-C₄ alkyl, typically methyl or ethyl, and most typically methyl;
to form an ethoxylated amine of the formula (III):

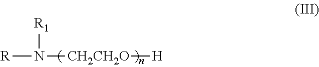

wherein R and R¹ have the meanings given above; and n represents at least 8, typically at least 9, and most typically at least 10, and at most 25, typically at most 20, and most typically at most 17; and
(b) oxidizing the ethoxylated amine of the formula (III) to give the N-oxide of formula (I).

In various non-limiting embodiments, all values and ranges thereof, both whole and fractional, including and between those set forth above, are hereby expressly contemplated for use herein.

The preparation of the ethoxylated amine of the formula (III) is well-known in the art. Its preparation is described, for instance, in U.S. Pat. No. 8,709,169 and European Patent No. 1 838 826, both mentioned above, where the ethoxylated amine is prepared as an intermediate to quaternary ammonium compound final products that can be utilized as hydrotropes. Both patents credit EP 0 90 117 A1 for a description of the ethoxylation reaction. The pertinent preparation teachings of all three documents are hereby incorporated herein by reference in various non-limiting embodiments.

In one embodiment, the secondary amine and ethylene oxide are charged to a reaction vessel. The ethylene oxide can be supplied to the reaction vessel randomly or in blocks. The amount of ethylene oxide necessary to obtain the desired degree of final product ethoxylation can be added all at once or sequentially over the time course of the reaction.

In one especially typical embodiment, the reaction vessel is initially charged with the total content of the secondary amine and a stoichiometric amount of ethylene oxide.

Later, additional ethylene oxide is introduced in the amount necessary to achieve the ultimate desired degree of ethoxylation.

Typically, the reaction temperature is equal to or greater than 40° C., typically equal to or greater than 80°° C., more typically equal to or greater than 100°° C., more typically equal to or greater than 120° C. In various non-limiting embodiments, all values and ranges thereof, both whole and fractional, including and between those set forth above, are hereby expressly contemplated for use herein.

In a more typical embodiment, the reaction temperature is kept between 100° C.-200° C. In various non-limiting embodiments, all values and ranges thereof, both whole and fractional, including and between those set forth above, are hereby expressly contemplated for use herein.

Pressure should be monitored during the reaction so that the maximal pressure does not exceed 5 bar, and typically does not exceed 4.7 bar, most typically does not exceed 4.5 bar. In various non-limiting embodiments, all values and ranges thereof, both whole and fractional, including and between those set forth above, are hereby expressly contemplated for use herein.

Having obtained the ethoxylated amine of the formula (III), this intermediate can be converted to the final product of formula (I) by reaction with hydrogen peroxide. Methods for oxidizing tertiary amines with hydrogen peroxide are well-known in the prior art. See, for example, U.S. Pat. No. 6,455,735 and the prior documents discussed therein.

In a typical embodiment, the ethoxylated amine of formula (III), hydrogen peroxide, and a chelating agent, for example, ethylenediaminetetraacetic acid (EDTA) or a salt thereof, for instance, the disodium salt, or, alternatively, one of the other chelating agents mentioned hereinbelow, are reacted in a reaction vessel at a temperature between 50° C.-100° C., most typically 55° C.-80° C. In various non-limiting embodiments, all values and ranges thereof, both whole and fractional, including and between those set forth above, are hereby expressly contemplated for use herein.

In an especially typical embodiment, the ethoxylated amine of formula (III) and the chelating agent are introduced to a reaction vessel along with water or other suitable solvent, and the temperature is manipulated to between 55° C.-65° C. Once the target temperature is reached, the hydrogen peroxide can be dosed into the reaction mixture and the temperature raised, for example, to around 70° C. If the solvent is water alone or in combination with a co-solvent, such as MPG (monopropylene glycol) or glycerol, there is no post-reaction work-up necessary and it is also not necessary to remove the solvent. On the other hand, it is also possible to carry out this reaction in lower alcohols, for example, methanol, ethanol, isopropyl alcohol etc., or in a mixture of such lower alcohols and water. However, these lower alcohol solvents, being volatile, are considered VOC and need to be removed. Other non-volatile solvents that can be used are other glycols such 1,3-propane diol, butane diols, diethylene glycols, dipropylene glycol etc., again, alone or in admixture with water. The use of water mixed with MPG is most typical, followed by the use of water mixed with glycerol. The amount of solvent is typically 0-80 wt % of the reaction mixture, most typically 40-60 wt %. In one typical embodiment, the solvent is removed from the product. In another typical embodiment, the solvent is not removed and is, therefore, present in the final product. In various non-limiting embodiments, all values and ranges thereof, both whole and fractional, including and between those set forth above, are hereby expressly contemplated for use herein.

Typically, catalysts are not needed, but the use of suitable catalysts is also contemplated.

In a particularly typical embodiment, the reaction is ideally carried out in a mixture of water+monopropylene glycol (MPG) as solvents. The solvents are not removed and the product once made is used “as is,” typically as a 50-60 wt % active in water+MPG. The reaction can also be carried out only in water or using water +glycerol. As the viscosity using water+glycerol is normally high, these are not the most typical unless they are diluted further to 40 wt % active. Instead of MPG, the reaction may also be carried out using other water miscible lower carbon chain length alcohols (which have VOC issues) or other diols or liquid polyols such as for example bio-based propane-1,3-diol, PEG or PPG. By using bio-based MPG or bio-based propane-1,3-diol, it will be possible to achieve higher RCI (Renewable Carbon Content) of the product. The main RCI comes from the biobased secondary amine but it is possible to add RCI of the solvents also to the product. In a similar vein, it is also possible to increase the RCI by using Bio-EO to make the alkyl amine ethoxylate. The use of ethylene oxide made from bio-based ethanol can increase RCI to close to 90%. In various non-limiting embodiments, all values and ranges thereof, both whole and fractional, including and between those set forth above, are hereby expressly contemplated for use herein.

The active % of the product is typically 20-100%, most typically 40-60%. In various non-limiting embodiments, all values and ranges thereof, both whole and fractional, including and between those set forth above, are hereby expressly contemplated for use herein.

Cleaning formulations containing the disclosed compounds are useful for a variety of cleaning purposes, for example, they can be formulated for, household cleaning, industrial cleaning, all-purpose cleaning, car washing, acidic and caustic cleaning, deck and floor cleaning, hard surface cleaning, metal cleaning, food & beverage cleaning, automated and manual dishwash, laundry detergents and the like.

For such purposes, the cleaning formulations may contain, in addition to the disclosed compounds of formula (I) and water, other conventional ingredients well known in the art of cleansing.

In a typical embodiment, the cleaning composition comprises one or more nonionic surfactants.

In a more typical embodiment, the one or more nonionic surfactants are selected from the group consisting of nonionic alkylene oxide adducts, especially C8-C18-linear and branched alcohol (alcohol alkoxylates) and amine alkoxylates comprising 1-20 ethyleneoxy units and 0-5 propyleneoxy units. The non-ionic alkyl polyglyceryl ethers made using C8-C18-linear and branched alcohol and 1-10 glycidol units or alkyl polyglycerylamines made using C8-C18-linear and branched alkyl amine and 1-10 glycidol units can also be used. In various non-limiting embodiments, all values and ranges thereof, both whole and fractional, including and between those set forth above, are hereby expressly contemplated for use herein.

The nonionic alkylene oxide adducts are well known conventional products wherein the molecule comprises a hydrophobic moiety and a moiety containing alkyleneoxy units, said latter moiety having a hydrophilic character. Thus the disclosure relates to the use of compounds of formula (I) as hydrotropes for nonionic surfactants in aqueous solutions. In other words, the disclosure relates to the improved solubilization of nonionic surfactants to make compositions with a good cleaning performance wherein water, a nonionic surfactant, a compound having the formula (I) as defined above, and other optional ingredients are combined and/or mixed in one or several steps.

The amounts of the components are suitably:
a) at least 0.05% by weight, typically at least 0.5% by weight, and at most 20% by weight, typically at most 15% by weight, and most typically at most 10% by weight, of alcohol alkoxylate,
b) at least 0.02% by weight, typically at least 0.1% by weight, and at most 20% by weight, typically at most 15% by weight, and most typically at most 10% by weight, of compound of formula (I), and
c) 0% by weight, typically at least 0.05% by weight, and at most 30% by weight, typically at most 20% by weight, more typically at most 15% by weight, and most typically at most 10% by weight, of alkali hydroxides, alkaline builders and/or alkaline complexing agents. In various non-limiting embodiments, all values and ranges thereof, both whole and fractional, including and between those set forth above, are hereby expressly contemplated for use herein.

It is especially typical that the compositions contain alkali hydroxides, alkaline builders and/or alkaline complexing agents.

The nonionic surfactants typically have the formula (IV):

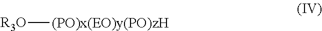

wherein R₃ is a C8 to C18 linear or branched alkyl group, typically C8 to C12; PO is a propyleneoxy unit, EO is an ethyleneoxy unit, x=0-5, typically 0-4, and most typically 0-2; y=1-20, typically 1-12, more typically 2-8, and most typically 2-5; and z =0-5, typically 0-4, more typically 0-2, and most typically 0. Thus, in addition to the 1-20 ethyleneoxy units, the C8-C18-alcohol alkoxylates may also contain up to 5 propyleneoxy units. The number of propyleneoxy units, when present, may be as small as 0.1 mole PO per mole alcohol. The ethyleneoxy units and the propyleneoxy units may be added randomly or in blocks. The blocks may be added to the alcohol in any order. The alkoxylates may also contain an alkyl group with 1-4 carbon atoms in the end position. Typically, the alkoxylates contain 2-8 ethyleneoxy units and 0-2 propyleneoxy units. The alkyl group of the nonionic surfactants may be linear or branched, saturated or unsaturated. Suitable linear nonionic surfactants are C9-C11 alcohol+4, 5, 6, 7 or 8 moles of EO, C8-C10 alcohol+3, 4, 5, 6, 7 or 8 moles of EO, C12-C14 alcohol+3, 4, 5, 6, 7 or 8 moles of EO and C10-C14 alcohol+8 moles of EO+2 moles of PO. Suitable branched nonionic surfactants are 2-ethylhexanol+3, 4 or 5 moles of EO, 2-ethylhexanol+2 moles of PO+4, 5 or 6 moles of EO, 2-propylheptanol+3, 4, 5 or 6 moles of EO and 2-propylheptanol+1 mole of PO+4 moles of EO, C9 or C11 alcohol+4, 5, 6, 7 or 8 moles of EO, tridecyl alcohol+4, 5, 6, 7 or 8 moles of EO,. Another example is 2-butyloctanol+5, 6 or 7 moles of EO. Wherever the degree of alkoxylation is discussed, the numbers represent molar average numbers. In various non-limiting embodiments, all values and ranges thereof, both whole and fractional, including and between those set forth above, are hereby expressly contemplated for use herein.

The compositions may be acidic, neutral or alkaline. Alkaline compositions are typically based on alkali hydroxides, alkaline builders and/or complexing agents. The alkaline compositions are especially typical.

The alkali hydroxides typically are sodium or potassium hydroxide. The alkaline builders may be an alkali carbonate or an alkali hydrogen carbonate, such as sodium carbonate, potassium carbonate, sodium hydrogen carbonate or potassium hydrogen carbonate, an alkali salt of a silicate, such as sodium silicate or sodium metasilicate, or alkali salts of phosphates, such as sodium orthophosphate. Alkaline builders that act through complexation are, e.g., sodium pyrophosphate and sodium tripolyphosphate and the corresponding potassium salts. The builder/complexing agent may also be organic. Examples of organic builders/complexing agents are aminocarboxylates, such as Glutamic acid, N,N-diacetate (GLDA), Methylglycine, N,N-diacetate (MGDA), sodium nitrilotriacetate (Na3NTA), sodium ethylenediamine tetraacetate (EDTA), sodium diethylenetriamine pentaacetate, sodium 1,3-propylenediamine tetraacetate, and sodiumhydroxyethylethylenediamine triacetate; aminopolyphosphonates, such as nitrilotrimethylene phosphonate; organic phosphates; polycarboxylates, such as citrates; and alkali salts of gluconic acid, such as sodium or potassium gluconates.

In neutral and acidic compositions complexing and/or pH adjusting agents may also be added, such as citric acid, oxalic acid, acetic acid, sulfamic acid, hydrochloric acid.

In another typical embodiment, the cleaning composition comprises one or more chelates.

In one embodiment, the chelate is at least one aminocarboxylate chelate selected from the group consisting of methylglycinediacetic acid (MGDA), N,N-dicarboxymethyl glutamic acid (GLDA), N-hydroxyethyliminodiacetic acid, nitrilotriacetic acid (NTA), ethylenediaminetetraacetic acid (EDTA), N-hydroxyethyl-ethylenediaminetriacetic acid (HEDTA), diethylenetriaminepentaacetic acid (DTPA), ethylenediaminetetraproprionic acid triethylenetetraaminehexaacetic acid (TTHA), tetracetyl ethylene diamine (TAED), iminodisuccinic acid (IDS), ethanol diglycine (EDG), and the respective alkali metal, ammonium and substituted ammonium salts thereof. In a particularly typical embodiment, the aminocarboxylate chelate is selected from the group consisting of EDTA, GLDA, MGDA, salts thereof, and combinations thereof.

In another embodiment, the chelate is a non-aminocarboxylate chelate containing carboxylate functionality but not a nitrogen atom. In a typical embodiment, the non-aminocarboxylate chelate is a divalent or higher valency carboxylic acid. In an especially typical embodiment, the non-aminocarboxylate chelate is at least one member selected from the group consisting of citric acid, isocitric acid, 2,3 hydroxycitric acid, tricarballylic acid, ethanetricarboxylic acid (HETA), aconitic acid, succinic acid, maleic acid, fumaric acid, oxaloacetic acid, ketoglutaric acid, butanetetracarboxylic acid, polycarboxylic acid, and the respective alkali metal, ammonium and substituted ammonium salts thereof. In a particularly typical embodiment, the non-aminocarboxylate chelate is selected from the group consisting of citric acid and salts thereof.

When one or more chelates are present in the formulation, they are present in a total combined chelate amount of greater than 0% by weight, typically at least 0.05% by weight, most typically at least 1% by weight, and at most 30% by weight, typically at most 20% by weight, more typically at most 15% by weight, and most typically at most 10% by weight. In various non-limiting embodiments, all values and ranges thereof, both whole and fractional, including and between those set forth above, are hereby expressly contemplated for use herein.

In another typical embodiment, in addition to one or more nonionic surfactants and/or chelates, the cleaning composition further comprises one or more adjunct ingredients selected from the group consisting of aesthetic agents, anti-filming agents, anti-redeposition agents, anti-spotting agents, anti-graying agents, beads, binders, biocides, bleach activators, bleach catalysts, bleach stabilizing systems, bleaching agents, brighteners, buffering agents, builders, carriers, clay, color speckles, control release agents, corrosion inhibitors, dish care agents, disinfectants, dispersant agents, draining promoting agents, drying agents, dyes, dye transfer inhibiting agents, enzymes, enzyme stabilizing systems, fillers, free radical inhibitors, fungicides, germicides, hydrotropes other than those of formula (I), opacifiers, perfumes, pH adjusting agents, pigments, processing aids, silicates, soil release agents, suds suppressors, anionic surfactants, cationic surfactants, stabilizers, thickeners, zeolite, and mixtures.

When one or more adjunct ingredients are present in the formulation, they are present in a total combined adjunct ingredient amount of greater than 0% by weight, typically at least 0.05% by weight, most typically at least 1% by weight, and at most 30% by weight, typically at most 20% by weight, more typically at most 15% by weight, and most typically at most 10% by weight. In various non-limiting embodiments, all values and ranges thereof, both whole and fractional, including and between those set forth above, are hereby expressly contemplated for use herein.

The present disclosure contemplates both diluted and concentrated

compositions.

Diluted compositions of the present disclosure are clear and stable. The clarity interval suitably is between 0-40° C., typically between 0-50° C., and most typically between 0-60° C. This may be adapted by changing the ratio of hydrotrope to nonionic surfactant. The diluted compositions normally contain at least 80% by weight of water, suitably at least 90% by weight, and normally at most 99.5% by weight of water, suitably at most 98% by weight. In various non-limiting embodiments, all values and ranges thereof, both whole and fractional, including and between those set forth above, are hereby expressly contemplated for use herein.

Concentrated compositions of the present disclosure are clear and stable. The clarity interval suitably is between 0-40° C., typically between 0-50° C., and most typically between 0-60° C. This may be adapted by changing the ratio of hydrotrope to nonionic surfactant. The concentrate normally contains at least 50% by weight of water, suitably at least 70% by weight, and normally at most 95% by weight of water, suitably at most 90% by weight. In various non-limiting embodiments, all values and ranges thereof, both whole and fractional, including and between those set forth above, are hereby expressly contemplated for use herein.

There are several advantages connected with the use of the compounds of formula (I) as hydrotropes for nonionic surfactants. Firstly, they are excellent hydrotropes that also contribute to the cleaning performance of the compositions. Their cleaning efficiency is very good even at high dilutions of the compositions and they perform just as well as the BEROL® R648 NG (Nouryon) when used in compositions for cleaning hard surfaces. Also, they are readily biodegradable and are expected to have low/no toxicity.

The disclosure will now be described in greater detail with reference to the following non-limiting examples.

### EXAMPLES

### Example 1: C12/14 Alkyl Methylamine Ethoxylate N-Oxide

A C12/14 alkyl methylamine ethoxylate N-Oxide can be prepared according to the following synthesis scheme:

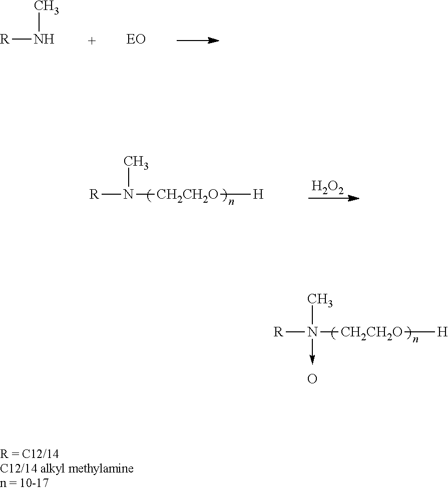

In greater detail, C12/14 alkyl methylamine ethoxylate N-oxide can be prepared as follows:

### Ethoxylation Reaction

To 265.2 g (1.27 moles) of monomethyl mono-(C12-C14-alkyl) amine, heated at 170° C. in a stainless steel autoclave that had been evacuated, 57.0 g (1.27 moles) of ethylene oxide were added with stirring during a period of 40 minutes. The temperature was kept at 170° C. during the addition, and the maximal pressure was 4.5 bar a. After the addition, the reaction mixture was kept at this temperature for 1 h. Then the temperature was lowered to 100° C., and 0.8 g KOH dissolved in methanol was added. The methanol and water were evaporated off at approximately 0.2 bar a at a temperature of 100-170° C., after which ethylene oxide was added at 170° C. in the appropriate amount to obtain the desired degree of ethoxylation. The maximal pressure during the addition was 4.5 bar a, and after the addition the reaction mixture was kept at this temperature until a steady pressure was obtained.

### Oxidation Reaction

289 g of the ethoxylated amine prepared above is charged along with 0.595 liters of water to a reaction vessel equipped with stirrers and maintained at a temperature of 60° C. Once the stirrers are started, 0.6 g of Na₂H₂EDTA are introduced to the reaction mixture. When the water, ethoxylated amine, and Na₂H₂EDTA are charged, and the temperature of the mixture is 60° C., the first dosing of H₂O₂ can be started. Mixing of the reactants is continued for one hour, then the temperature is increased 65° C. and then a second dosage of H₂O₂ is started. Mixing of the reactants is continued for another one hour, then the temperature is increased to 70° C. and then the reaction is continued for twelve hours.

Samples are taken from the reaction mixture for analysis and correction if needed. If the H₂O₂ content is too high, this is corrected with the addition of ethoxylated amine. If the free amine content is too high, this is corrected with the addition of H₂O₂. Samples should be taken at a three hour intervals and corrections made at each sampling as necessary until no further corrections are needed.

The batch on pH can be adjusted as desired with H₂SO₄. The reaction mixture should continue to be stirred for 15 minutes after each addition.

Once a final sample of the reaction mixture has been taken, and the desired attributes of the product have been confirmed, the product mixture is cooled down to 30° C. and the temperature block turned off.

The product can be discharged from the reactor into the desired packaging using a tap system, or the product can be pumped into a storage tank.

### Example 2

In an analogous manner, hydrotropes can be made from the following combinations of secondary amines and ethylene oxide:

| Secondary Amine | Ethylene Oxide (moles) |
|---|---|
| n-decylmethylamine | 8 |
| laurylmethylamine | 10 |
| C12/14 alkylmethylamine | 12 |
| cocoalkylmethylamine | 14 |
| tallowalkylmethylamine | 16 |
| soyaalkylmethylamine | 18 |

### Example 3

BEROL® R648 NG (Nouryon) is a market leading product for cleaning formulations that affords best-in-class cleaning, from a renewable, vegetable source with low or no environmental persistence and ecotoxicity and ideally non-label as-sold. Though BEROL® R648 NG is readily biodegradable, it has certain drawbacks such as it is a quat, and has low RCI (Renewable Carbon Index) of 29%. With the fast changing regulations and environmental awareness, the availability of an EO-free (and dioxane free) non-quat product with higher RCI (typically >50%) that performs as well as BEROL® R648 NG is desired.

C12/14 alkyl methylamine ethoxylate N-Oxide meets all the criteria mentioned above with performance as good as the benchmark BEROL® R648 NG. C12/14 alkyl methylamine ethoxylate N-Oxide is readily biodegradable with high RCI, expected to exhibit low/no toxicity, and its cleaning performance is as good as the benchmark BEROL® R648 NG.

The suitability of C12/14 alkyl methylamine ethoxylate N-Oxide to solubilize BEROL® 260 (C9-C11 alcohol ethoxylate) and the cleaning power of the resultant formulations were tested in comparison to BEROL® R648 NG. Test formulations A, B, and C in Table 1 comprised 5% BEROL® 260, 8% DISSOLVINE® GL-47-S, and either BEROL® R648 NG or C12/14 alkyl methylamine ethoxylate N-Oxide in water (formulation A) or water and MPG (formulation C) to reach a cloud point of 40-50° C. for the final formulation. Test formulations D, E, and F in Table 2 comprised 5% BEROL® 260, 8% DISSOLVINE® GL-47-S, 2% NaOH, and either BEROL® R648 NG or C12/14 alkyl methylamine ethoxylate N-Oxide in water (formulation D) or water and MPG (formulation F) to reach a cloud point of 30-40° C. for the final formulation.

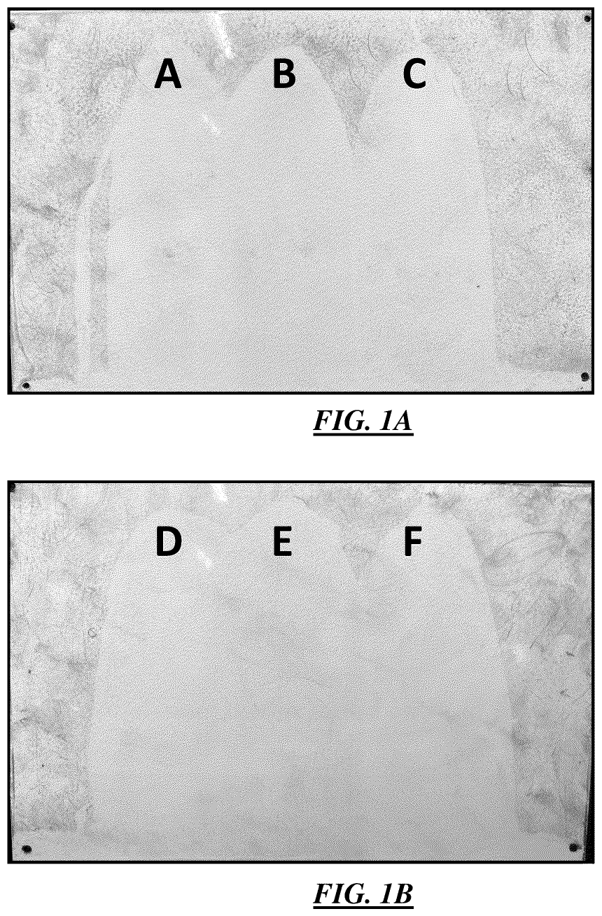

The degreasing effect of the test formulations was assessed on a white painted metal plate soiled uniformly with a tough-to-clean greasy soil collected from a train engine. The cleaning formulation (1:40 dilution in water) was applied simply by pouring it over the surface (non-mechanical cleaning). After a short time interval, the whole plate was rinsed with tap water and cleaning performance was assessed quantitatively. The results are tabulated below and depicted in FIG. 1A (for the respective cleaning formulations A, B, and C containing no NaOH) and FIG. 1B. (for the respective cleaning formulations D, E, and F containing 2% NaOH).

| | | | | Cloud | | Appearance |
|---|---|---|---|---|---|---|
| | | | | Point | Cleaning | (Room |
| Formulation | Hydrotrope | Active % | W/W | (° C.) | Performance | Temp) |
| A (no NaOH) | Inventive | 2.76 | 4.5 | 47 | Good | Clear |
| B (no NaOH) | Comparison | 1.92 | 3.2 | 47 | Good | Clear |
| C (no NaOH) | Inventive | 2.76 | 4.5 | 47 | Good | Clear |
| D (2% NaOH) | Inventive | 2.76 | 4.5 | 28 | Good | Clear |
| E (2% NaOH) | Comparison | 2.04 | 3.4 | 37 | Good | Clear |
| F (2% NaOH | Inventive | 2.76 | 4.5 | 30 | Good | Clear |

The data in the foregoing table and in FIG. 1A and FIG. 1B show the inventive hydrotrope (C12/14 alkyl methylamine ethoxylate N-Oxide) in water or water and MPG performs as well as the comparison hydrotrope (BEROL® R648 NG) in the absence or presence of NaOH in the cleaning formulation.

While the present disclosure has been described in conjunction with the specific embodiments set forth above, many alternatives, modifications and other variations thereof will be apparent to those of ordinary skill in the art. All such alternatives, modifications and variations are intended to fall within the spirit and scope of the present disclosure.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

What is claimed is:

1. A compound of the formula (I): 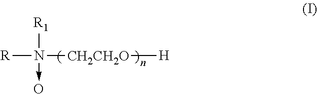
wherein
R represents a fatty linear or branched, saturated or unsaturated alkyl group having 8-30 carbon atoms;
R¹ represents a linear or branched, saturated or unsaturated lower alkyl group having 1-8 carbon atoms; and
n represents at least 8 and at most 25.

2. The compound of the formula (I) according to claim 1, wherein:
R represents C₈-C₂₂ alkyl or alkenyl;
R¹ represents C₁-C₄ alkyl; and
n represents at least 9 and at most 20.

3. The compound of the formula (I) according to claim 1 wherein:
R represents C₁₀-C₁₈ alkyl or alkenyl; and
R¹ represents methyl or ethyl; and
n represents at least 10 and at most 15.

4. The compound according to claim 1, which is derived from a secondary amine of the formula (II): 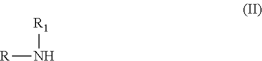
wherein
R represents a fatty linear or branched, C₈-C₂₀ alkyl or alkenyl; and
R¹ represents a linear or branched, C₁-C₄ alkyl.

5. The compound according to claim 4, wherein the secondary amine is chosen from octylmethylamine, laurylmethylamine, cocoalkylmethylamine, n-decylmethylamine, tallowalkylmethylamine, soyaalkylmethylamine, and C12/14alkylmethylamine.

6. The compound according to claim 1, which has the formula (Ia): 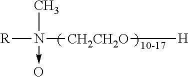
wherein
R represents a fatty linear or branched, saturated or unsaturated alkyl group having 12 or 14 carbon atoms.

7. A process for preparing a compound according to claim 1, comprising the steps of
(a) ethoxylating an amine of the formula (II): 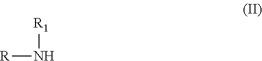
wherein
R represents a fatty linear or branched, saturated or unsaturated alkyl group having 8-30 carbon atoms; and
R¹ represents a linear or branched, saturated or unsaturated lower alkyl group having 1-8 carbon atoms;
to form an ethoxylated amine of the formula (III): 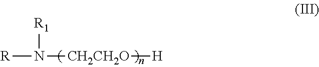
wherein R and R¹ have the meanings given above; and n represents at least 8; and
(b) oxidizing the ethoxylated amine of the formula (III) to give the N-oxide of formula (I).

8. An aqueous cleaning composition comprising:
(a) at least one compound according to claim 1; and
(b) water.

9. The aqueous cleaning composition according to claim 8, which further comprises one or more nonionic surfactants.

10. The aqueous cleaning composition according to claim 9, wherein the more or more nonionic surfactants are chosen from nonionic surfactants having the formula (IV): 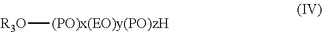
wherein
R₃ is a C8 to C18 linear or branched alkyl group;
PO is a propyleneoxy unit;
EO is an ethyleneoxy unit;
x=0-5;
y=1-20; and
z=0-5;

11. The aqueous cleaning composition according to claim 8, which further comprises one or more chelates.

12. The aqueous cleaning composition according to claim 8, which further comprises one or more adjunct ingredients chosen from aesthetic agents, anti-filming agents, anti-redeposition agents, anti-spotting agents, anti-graying agents, beads, binders, biocides, bleach activators, bleach catalysts, bleach stabilizing systems, bleaching agents, brighteners, buffering agents, builders, carriers, clay, color speckles, control release agents, corrosion inhibitors, dish care agents, disinfectants, dispersant agents, draining promoting agents, drying agents, dyes, dye transfer inhibiting agents, enzymes, enzyme stabilizing systems, fillers, free radical inhibitors, fungicides, germicides, hydrotropes other than those of formula (I), opacifiers, perfumes, pH adjusting agents, pigments, processing aids, silicates, soil release agents, suds suppressors, anionic surfactants, cationic surfactants, stabilizers, thickeners, zeolite, and mixtures thereof.

13. A method of cleaning an object to be cleaned comprising the step of contacting the object with the aqueous cleaning composition according to claim 8.
